(19) United States
(12) Patent Application Publication
(10) Pub. No.: US 2025/0194911 A1
(43) Pub. Date: Jun. 19, 2025

(54) ENDOSCOPIC INSTRUMENT, AND METHOD AND SYSTEM FOR DETERMINING LESION LOCATION BY USING SAME

(71) Applicant: XAIMED CO., LTD, Seoul (KR)

(72) Inventors: Sang Min PARK, Seoul (KR); Sei Hoon PARK, Seoul (KR)

(73) Assignee: XAIMED CO., LTD, Seoul (KR)

(21) Appl. No.: 18/288,806

(22) PCT Filed: Apr. 29, 2022
(86) PCT No.: PCT/KR2022/006143

§ 371 Date: May 23, 2024

(30) Foreign Application Priority Data
Apr. 29, 2021 (KR) 10-2021-0055671
May 3, 2021 (KR) 10-2021-0057088

(51) Int. Cl. A61B 1/07; A61B 1/00

(52) U.S. Cl. CPC A61B 1/07; A61B 1/00032; A61B 1/00087

(57) ABSTRACT

The present application provides an endoscope instrument comprising: a main body which is provided at the top end of an endoscope device, and which can be operated by the operation of an operator; and a light guide unit, which is provided at the main body to emit light forward.

### TECHNICAL FIELD

The present disclosure relates to an endoscopic tool, a method and system for determining the location of lesions using the endoscopic tool, more specifically, to the endoscopic tool capable of accurately identifying the target point by illuminating light to the front of the endoscopic tool and an endoscope system capable of accurately recognizing and displaying the position of the target by measuring the distance between the endoscopic tool and the target during the procedure.

### BACKGROUND ART

An endoscope is a device that inserts a camera directly into the body of animals such as humans to directly view the internal organs. Depending on the test subject, the endoscope examinations include gastroscopy, colonoscopy, and cystoscopy. The size, shape, and location of the lesion can be evaluated while directly observing the lesion through the endoscope, and a biopsy can be performed at the same time.

Recently, with the advancement of medical technology, the procedure is performed by inserting surgical instruments such as forceps, clamps, scissors, etc. that can be operated by the operator from the outside along with the endoscope. In particular, during tissue examination, surgical instruments such as forceps are used to extract or excise the lesion tissue to be examined through an endoscope channel.

In this process, the target point that guides the surgical position of the surgical instrument and the location of the lesion tissue do not match, which often causes wounds to the inner wall of the test object. In severe cases, there was a problem in which perforations can occur in the inner wall of the test object.

Therefore, a method to solve these problems is required.

### DESCRIPTION OF EMBODIMENTS

### Technical Problem

In order to solve the problems of the prior art described above, the present disclosure is to provide an endoscopic tool that allows the extraction or resection of lesion tissue by accurately identifying the location of the lesion during tissue examination using an endoscope device.

In addition, the present disclosure is to provide a method for determining the location of a lesion and an endoscope system that can accurately determine the location of the lesion and extract or excise the lesion tissue during a tissue examination using an endoscope device.

The problems of the present disclosure are not limited to the problems mentioned above, and other problems not mentioned will be clearly understood by those skilled in the art from the description below.

### Solution to Problem

In one aspect of the present disclosure, an endoscopic tool provided at the distal end of an endoscope device comprises a main body capable of operating by an operator and a light guide unit provided at the main body to emit light in a direction in which the main body moves.

In one aspect of the present disclosure, an endoscope system comprises an endoscope device having a tube in which a camera is placed in one of a plurality of channels formed inside the endoscope device, an endoscopic tool having a light guide unit that is inserted into another channel among the plurality of channels and irradiates a plurality of lights so that at least two light spots are focused on a surface of a tissue inside a human body and a controller measuring a distance from a tip of the endoscopic tool to the surface of the tissue according to a distance between light spots formed on the surface of the tissue based on a preset distance from the tip of the endoscopic tool to the surface of the tissue according to the distance between light spots.

In another aspect of the present disclosure, an endoscope system comprises an endoscope device having a tube in which a camera is placed in one of a plurality of channels formed inside the endoscope device, an endoscopic tool having a light guide unit that is inserted into another channel among the plurality of channels and irradiates light so that a light spot is focused on a surface of a tissue inside a human body and a controller measuring a distance from a tip of the endoscopic tool to the surface of the tissue according to a size of the light spot formed on the surface of the tissue based on a preset distance from the tip of the endoscopic tool to the surface of the tissue according to the size of the light spot.

In one aspect of the present disclosure, a method for determining a location of a lesion comprises forming a light spot on a surface of a tissue by irradiating light to the tissue inside a human body using a light guide unit mounted on an endoscopic tool and measuring a distance between a tip of the endoscopic tool and the lesion of the tissue based on information of a preset light spot.

### Advantageous Effects of Disclosure

According to the present disclosure, the endoscopic tool can be easily operated by the operator and accurately identify a location of the lesion during tissue examination using the endoscope device, thereby removing or resecting the lesional tissue.

In particular, according to embodiments of the present invention, a light guide unit can be provided in various shapes and locations, so that it can be flexibly applied to changing conditions, such as the type of procedure being performed or the endoscopic tool being used.

In addition, according to an embodiment of the present invention, an endoscope system for determining the location of a lesion can accurately measure a distance between an endoscope device and a surface of the tissue, and can stably perform a tissue examination using an endoscope device.

In addition, according to an embodiment of the present invention, an endoscope system for determining the location of a lesion can display the location of the endoscopic tool on the effective screen using a trace mark, allowing the operator to know the exact location of the endoscopic tool.

The effects of the present disclosure are not limited to the effects mentioned above, and other effects not mentioned will be clearly understood by those skilled in the art from the description of the claims.

### BRIEF DESCRIPTION OF DRAWINGS

FIG. 1 shows a diagram of an endoscope device according to embodiments of the present disclosure.

FIG. 2 and FIG. 3 show a diagram of an endoscopic tool according to a first embodiment of the present disclosure.

FIG. 4 shows a diagram of an endoscopic tool according to a second embodiment of the present disclosure.

FIG. 5 and FIG. 6 show a diagram of an endoscopic tool according to a third embodiment of the present disclosure.

FIG. 7 shows a diagram of a modified endoscopic tool according to the third embodiment of the present disclosure.

FIG. 8 shows a diagram of an endoscopic tool according to a fourth embodiment of the present disclosure.

FIG. 9 shows a schematic diagram of an endoscope system according to a first embodiment of the present disclosure.

FIG. 10 and FIG. 11 is a view showing a distance between light points depending on the distance between an optical fiber of the endoscopic tool and a surface of a tissue according to embodiments of the present disclosure.

FIG. 12 shows a schematic diagram of an endoscope system according to a second embodiment of the present disclosure.

FIG. 13 shows a schematic diagram of an endoscope system according to a third embodiment of the present disclosure.

FIG. 14 shows a schematic diagram of an endoscope system according to a fourth embodiment of the present disclosure.

FIG. 15 shows a schematic diagram of an endoscope system according to a fifth embodiment of the present disclosure.

FIG. 16 is a flowchart to explain a method for determining a location of a lesion using an endoscope system according to embodiments of the present disclosure.

### DETAILED DESCRIPTION

The following is a description of a preferred embodiment of the present invention, which can be used to specifically achieve the purpose of the present invention. In describing this embodiment, the same name and the same symbol are used for the same component, and additional explanation is omitted accordingly.

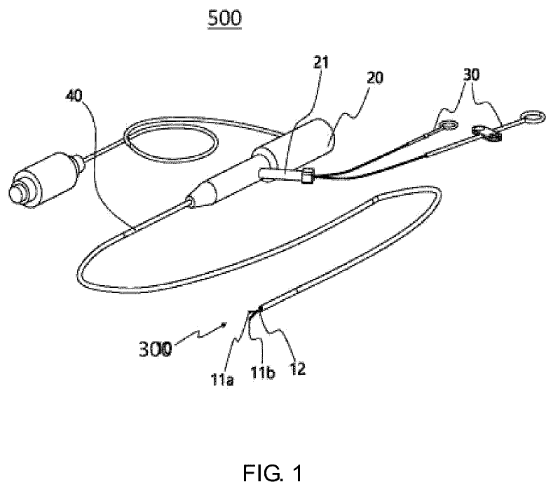

FIG. 1 is a diagram showing an endoscope device according to embodiments of the present disclosure.

As shown in FIG. 1, the endoscope device (500) comprises a main body (20), a biopsy channel (21) protruding from the main body (20), and an insertion tube (40) that is configured to be inserted into a human body.

The insertion tube (40) has a plurality of channels formed in a hollow form inside, and one of these channels has a camera (not shown) at a tip of the insertion tube, and the other channel is in a form connected to the biopsy channel (21).

The endoscopic tool (300) may be inserted into the biopsy channel (21) and moved along the channel connected to the biopsy channel (21) to perform its function in the form that is placed at the tip of the insertion tube (40). In other words, a surgeon can operate the endoscopic tool (300) through the manipulator (30) of the endoscopic tool (300) that is inserted into the biopsy channel (21).

Meanwhile, the endoscopic tool (300) may be applied to various tools such as forceps, snare, injector, clamp, and scissors, and the endoscopic tool (300) in this embodiment is exemplified as having a forceps shape.

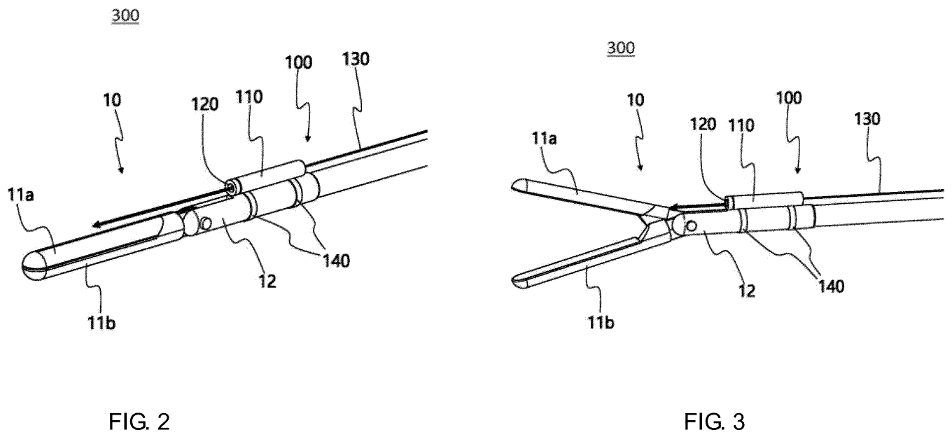

FIG. 2 and FIG. 3 show diagrams of an endoscopic tool according to embodiments of the present disclosure.

As shown in FIGS. 2 and 3, the endoscopic tool (300) comprises a body (10) and a light guide unit (100).

The body (10) is formed at the tip of the endoscope device, as described above, and is formed to be movable by the surgeon's operation. The body (10) may include a hinge part (12) that forms a rotation shaft, a first clip part (11a) that is fixed on the rotation shaft of the hinge part (12) and may be variably angled in the first direction, and a second clip part (11b) that is fixed on the rotation shaft of the hinge part (12) and may be variably angled in the second direction, which is the opposite direction of the first direction.

The light guide unit (100) is provided on the body (10) and irradiates light in the same direction as the direction of movement of the body while the body is moving toward a target. The light guide unit (100) may be applied to any means that can irradiate light in the direction of movement of the body, and may be provided in various positions on the body (10). In this embodiment, the light guide unit (100) is in the form of being provided on the hinge part (12) of the body (10).

In embodiment, the light guide unit (100) may include a light source (120) that generates light when supplied with power, a housing (110) that surrounds the light source (120) and is fixed in a predetermined position on the body (10), and a cable (130) that connects to one side of the light source (120) and connects to an external power source on the other side to supply power to the light source (120). The light guide unit (100) includes a power cable (130) to use an external power source to supply power to the light source (120), but it may have a structure that can be supplied with its own power by including a power source such as a battery inside the housing (110) without a power cable.

In this case, the housing (110) may be formed to be detachably mounted on the body (10) in various ways, and in this embodiment, a fixing means such as a band (140) may be used.

The light guide unit (100) may be placed in a position of the body (10) such that the light emitted from the light source (120) is blocked by the rotation of the first clip member (11a) or the second clip member (11b), as shown in FIG. 3. In other words, the light guide unit (100) may be placed in a position where the light is emitted forward through the light source (120) when the first clip member (11a) and the second clip member (11b) are maintained in a non-rotated state, as shown in FIG. 2, and in a position where the light emitted from the light source (120) can be blocked by the first clip member (11a) when the first clip member (11a) and the second clip member (11b) are rotated to an open state, as shown in FIG. 3.

When the light guide unit (100) is placed, as shown in FIG. 2, the light guide unit (100) may play a role of guide for setting the position of the target (e.g., lesion) when the operator uses the endoscopic tool to find the target point (e.g., lesion). As shown in FIG. 3, when the operator uses the endoscopic tool to remove or resect the target (lesion), the light emitted from the light guide unit (100) is reflected from the target, thereby preventing the operator from causing glare and performing the procedure stably.

In this way, the present embodiment can accurately identify the location of the lesion during tissue examination using an endoscope device by including the light guide unit (100) that is directly installed in the body (10) that is formed to be operable by the operator's operation, and thereby remove or resect the lesion of the tissue.

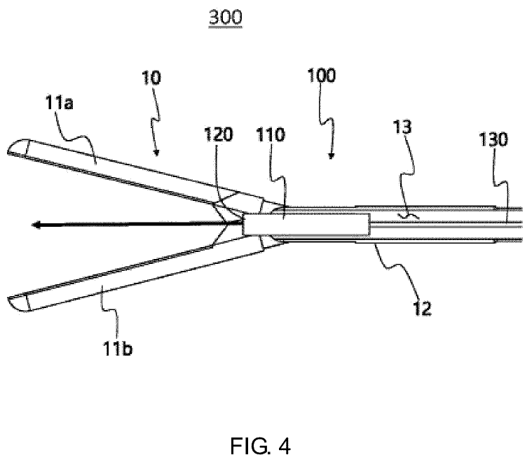

FIG. 4 shows a diagram of an endoscopic tool according to a second embodiment of the present disclosure.

As shown in FIG. 4, the endoscopic tool (300) may have the same components as the endoscopic tool shown in the first embodiment of the present disclosure. Therefore, the explanation of the same components is omitted.

In the embodiment of the present disclosure, a space (13) is formed on the inner side of the hinge part (12) of the endoscopic tool (300), and the light guide unit (100) of the endoscopic tool (300) may be provided in the space (13).

As such, when the first clip member (11a) and the second clip member (11b) are maintained in an open state with their angles variable in opposite directions, the light generated from the light source (120) of the light guide unit (100) can pass through the first clip member (11a) and the second clip member (11b) and be irradiated in the forward direction of the body (10).

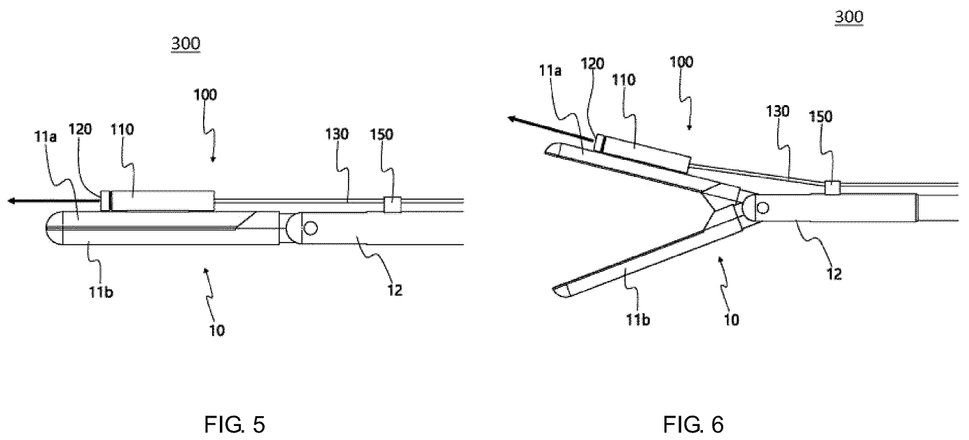

FIGS. 5 and 6 show diagrams of an endoscopic tool according to a third embodiment of the present disclosure.

As shown in FIGS. 5 and 6, the endoscopic tool (300) may have the same components as the endoscopic tool shown in the first embodiment of the present disclosure. Therefore, the explanation of the same components is omitted.

In the embodiment of the present disclosure, the light guide unit (100) of the endoscopic tool (300) may be provided in the first clip member (11a) or the second clip member (11b) instead of the hinge part (12) of the body (10).

As such, when the first clip member (11a) and the second clip member (11b) are maintained in a non-rotated state, as shown in FIG. 5, the light can be emitted forward through the light source (120). When the first clip member (11a) and the second clip member (11b) are rotated to an open state, as shown in FIG. 6, the light emitted from the light source (120) will proceed in the direction corresponding to the rotation angle of the first clip member (11a).

This arrangement of the light guide unit (100) can achieve the same effects as the first embodiment of the present disclosure.

In addition, in embodiments, a cable restraint unit (150) is provided on the hinge part (12) to prevent the power cable (130) from separating from the hinge part (12) when the first clip member (11a) and the second clip member (11b) are rotated to an open state.

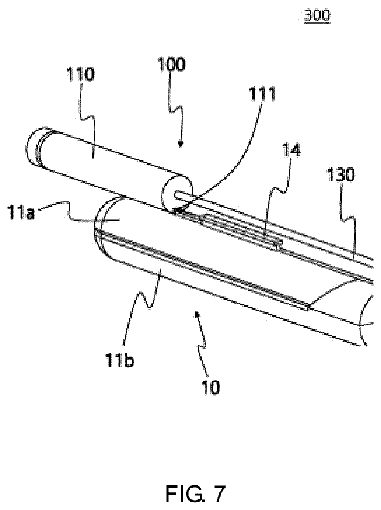

In addition, as shown in FIG. 7, the light guide unit (100) may be attached to the first clip member (11a) using a rail-mounting method.

Specifically, in embodiments, a guide rail (14) for connecting the light guide unit (100) is formed on the first clip member (11a), and a rail groove (111) capable of inserting the guide rail (14) is formed on the housing (110) of the light guide unit (100). Accordingly, the light guide unit (100) is easily detachable from the first clip member (11a). In other embodiments, the guide rail (14) may also be formed on the second clip member (11b).

The above mounting method can also be applied to the previously described embodiment in which the light guide unit (100) is placed on the hinge part.

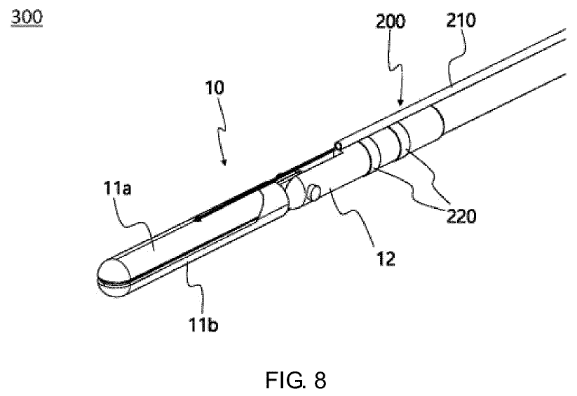

FIG. 8 shows a diagram of an endoscopic tool according to a fourth embodiment of the present disclosure.

As shown, the endoscopic tool (300) may have the same components as the endoscopic tool shown in the first embodiment of the present disclosure. Therefore, the explanation of the same components is omitted.

In embodiments of the present disclosure, the light guide unit (200) of the endoscopic tool (300) includes an optical fiber (210) that is connected to an external light source that generates light and is designed to be internally reflected and irradiated forward from the light source.

The optical fiber (210) may be placed on one side of the hinge part (12) of the endoscopic tool (300) and be placed along the longitudinal direction of the body (10). The optical fiber (210) is fixed by a fixing band (220) on the hinge part (12), but the fixing method of the optical fiber (210) may also be done in other ways.

Although not shown in the figure, in this embodiment of the present disclosure, the optical fiber (210) is housed inside the hinge part (12), so that the light emitted from the optical fiber (210) can be irradiated forward when the first clip member (11a) and the second clip member (11b) are rotated to an open state.

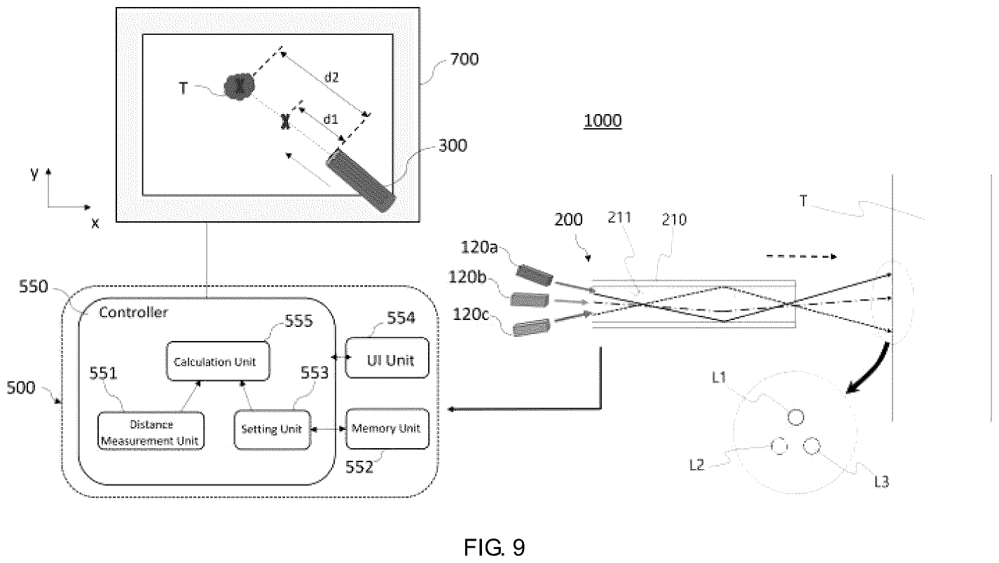

FIG. 9 shows a schematic diagram of an endoscope system according to the first embodiment of the present disclosure.

As shown, the first embodiment of the endoscope system (1000) according to the present disclosure includes an endoscopic tool (300), an endoscope device (500), and a display unit (700).

The endoscopic tool (300) may include a light guide unit (200) and is inserted into a tube included in the endoscope device (500) for operation by the operator. In embodiments, the light guide unit (200) is shown as a module that includes an optical fiber (210), and the description of the endoscopic tool (300) is omitted as it can have a variety of configurations similar to those described above.

In embodiments, the light guide unit (200) may include multiple light sources (120a, 120b, 120c) to irradiate multiple lights into the interior of the optical fiber (210), and as the operator operates the optical guide unit (200), multiple light points (L1, L2, L3) are formed on the surface of the tissue (e.g., lesion) inside the human body. Multiple light sources (120a, 120b, 120c) may be arranged at different angles to the input end of the optical fiber (210), or they may be arranged at parallel angles. Light emitted from multiple light sources (120a, 120b, 120c) is irradiated into the input end of the optical fiber (210) and emitted from the output end of the optical fiber (210) at a predetermined angle that is not parallel.

In embodiments, the optical fiber (210) that constitutes the optical guide unit (200) may be formed by multiple fibers, but it is preferable to be formed by a single fiber so that multiple light paths generated from the light source (120a, 120b, 120c) are independently formed to form multiple light points (L1, L2, L3) on the surface of the tissue. Multiple light sources (120a, 120b, 120c) may be of the same color or of different colors.

The endoscope device (500) may include mechanical/mechanically configured components of the aforementioned FIG. 1 and a control unit (550) that controls the entire endoscope system (1000) optically or electrically.

The control unit (550) may include a distance measurement unit (551), a setting unit (553), and a calculation unit (555). The distance measurement unit (551) calculates the distance between the multiple light points (L1, L2, L3) formed on the surface of the tissue by the light irradiated from the optical guide unit (200) included in the endoscopic tool (300). The distance between multiple light points varies depending on the distance between the endoscopic tool (i.e., the tip of the endoscopic tool) and the surface of the tissue inside the human body. The setting unit (555) pre-sets a standard distance between the endoscopic tool and the surface of the tissue inside the human body according to the distance between multiple light points, and this standard distance may be stored in a memory unit (552) as a lookup table. The calculation unit (555) may accurately calculate the distance between the endoscopic tool and the surface of the tissue by comparing the value of the standard distance set by the setting unit (555) to the value of the distance between the multiple light points formed on the surface of the tissue measured by the distance measurement unit (551) as the endoscopic tool (300) device moves.

Display unit (700) displays an image of the inside of the human body captured by the camera according to the movement of the endoscope device. At this time, the UI (User Interface) unit (554) of the endoscope device (500) may display a track mark (x) on the valid screen of the display unit (700) based on the distance between the endoscopic tool (300) and the tissue surface measured by the calculation unit (555) under the control of the control unit (550). For example, the track mark (x) may be the location point of the endoscopic tool (300) if the endoscopic tool (300) moves a first distance (d1). By displaying the track mark (x) on the valid screen, the operator can be made aware of whether the endoscopic tool (300) is accurately positioned on the surface of the target tissue (T). The track mark (x) may be displayed as a UI, but it may also be displayed as a two-dimensional coordinate value (x, y) for the entire valid screen of the display unit (700).

In this way, the endoscope system according to the present embodiment can easily grasp the distance between the endoscopic tool and the surface of the target tissue, so that the location of the lesion can be accurately grasped when using the endoscope device for tissue examination, and the lesion tissue can be removed or resected.

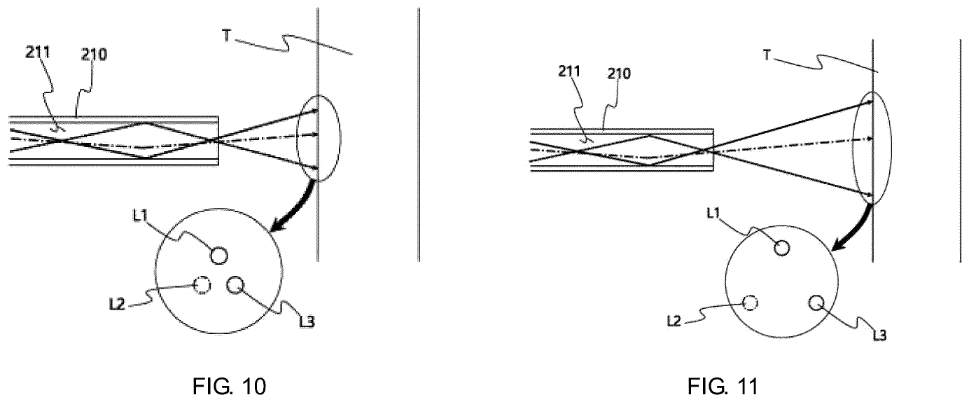

FIG. 10 and FIG. 11 is a view showing a distance between light points depending on the distance between an optical fiber of the endoscopic tool and a surface of a tissue according to embodiments of the present disclosure.

As shown, multiple light beams separated from each other generated from the light source are irradiated into the optical fiber (210), and different lights that have passed through the reflection space (211) inside the optical fiber (210) are irradiated to the outside of the optical fiber (210) and then reach the tissue (T) surface. As a result, multiple light spots (L1, L2, L3) are formed on the tissue surface, and the spacing distance between multiple light spots may vary depending on the spacing distance between the tip of the optical fiber (210) and the tissue (T).

For example, in the case of FIG. 11, where the spacing distance between the tip of the optical fiber (210) and the tissue (T) is farther than the spacing distance illustrated in FIG. 10, the spacing distance between the light spots formed by multiple lights (L1, L2, L3) will be further away than the spacing distance between the light spots formed by multiple lights (L1, L2, L3) in FIG. 10.

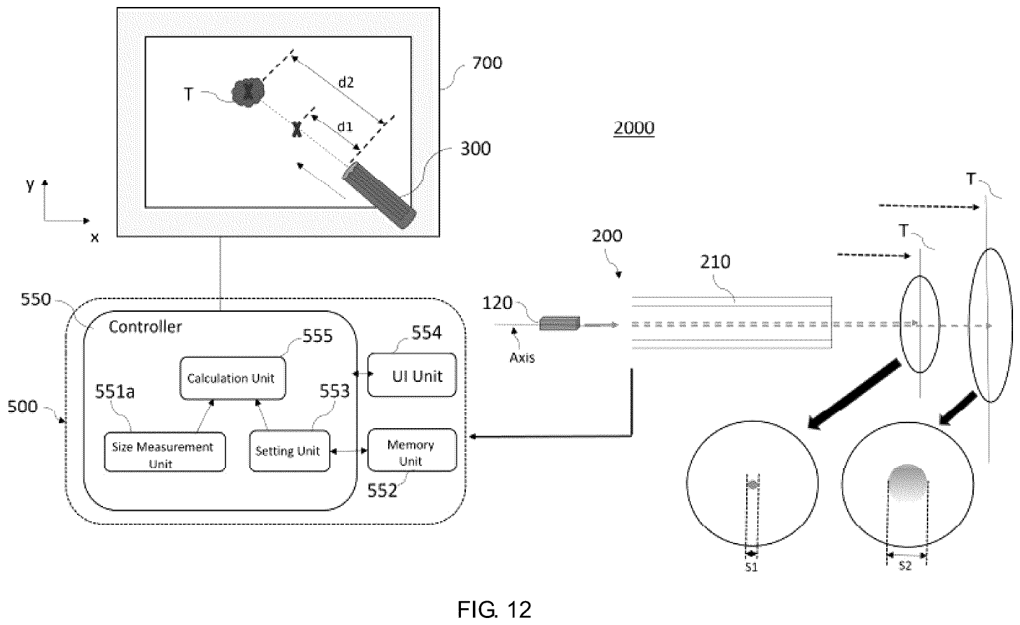

FIG. 12 shows a schematic diagram of an endoscope system according to a second embodiment of the present disclosure.

As shown, the endoscope system (2000) according to the second embodiment includes the same or similar components and effects as the endoscope system (1000) according to the first embodiment.

However, in the embodiment, the light guide unit (200) may include a single light source (120) for irradiating a single light into the interior of the optical fiber (210), and the optical fiber (210) may be composed of multiple fibers in a bundle form. The single light source (120) may be arranged parallel to the axial axis (Axis) of the optical fiber (210). The light guide unit (200) having such a structure forms a light spot on the surface of the tissue inside the human body as it is operated by the operator. The size (S1, S2) of the light spot formed on the tissue surface increases as the distance between the optical fiber and the tissue surface increases, and the brightness of the light spot decreases.

The control unit (550) may include a size measurement unit (551a), a setting unit (553), and a calculation unit (555). The size measurement unit (551a) calculates the size of the light spot formed on the tissue surface by the light irradiated from the light guide unit (200) included in the endoscopic tool (300). The size of the light spot varies depending on the distance between the endoscopic tool (i.e., the tip of the endoscopic tool) and the surface of the tissue inside the human body. The setting unit (555) pre-sets the standard distance between the endoscopic tool and the surface of the tissue inside the human body according to the size of the single light spot, and this standard distance may be stored in the memory unit (552) as a look-up table. The calculation unit (555) can accurately calculate the distance between the endoscopic tool and the tissue surface by comparing the value measured by the size measurement unit (551a) for the size of the light spot formed on the tissue surface according to the movement of the endoscopic tool with the pre-set standard distance value from the setting unit (555).

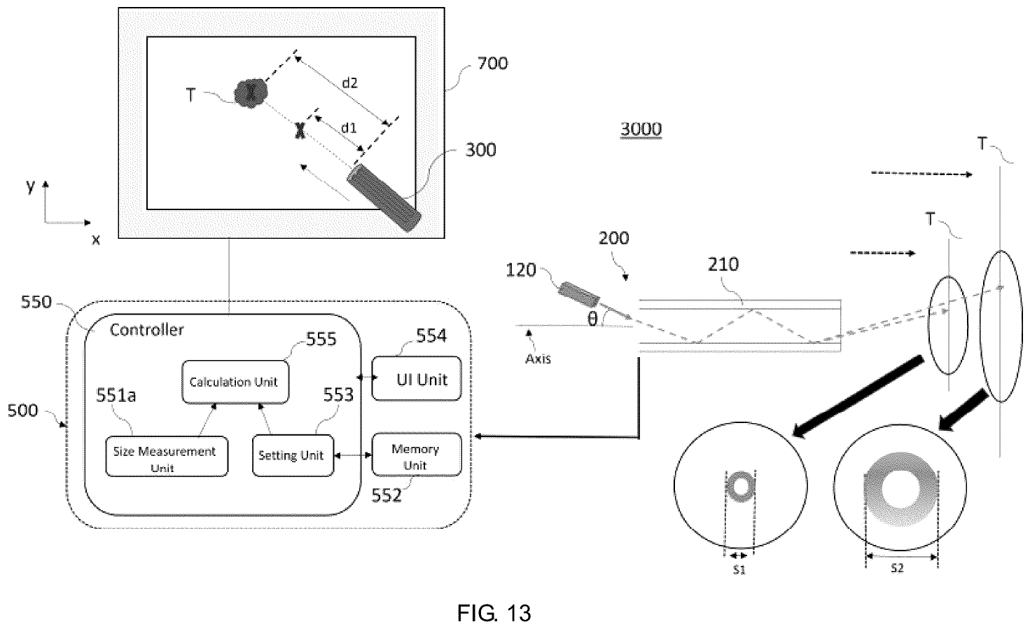

FIG. 13 shows a schematic diagram of an endoscope system according to a third embodiment of the present disclosure.

As shown, the endoscope system (3000) according to the third embodiment includes the same or similar components and effects as the endoscope system (1000) according to the first embodiment.

However, in the embodiment, the light guide unit (200) may include a single light source (120) for irradiating a single light into the interior of the optical fiber (210), and the optical fiber (210) may be composed of multiple fibers in a bundle form. In this case, the single light source (120) may be arranged at a predetermined angle (0) with respect to the axial axis (Axis) of the optical fiber (210). The light guide unit (200) having such a structure forms a donut-shaped light spot on the surface of the tissue inside the human body as it is operated by the operator. The size (S1, S2) of the donut-shaped light spot formed on the tissue surface increases as the distance between the optical fiber and the tissue surface increases, and the brightness of the donut-shaped light spot decreases.

The control unit (550) may include a size measurement unit (551a), a setting unit (553), and a calculation unit (555). The size measurement unit (551a) calculates the size of the donut-shaped light spot formed on the tissue surface by the light irradiated from the light guide unit (200) included in the endoscopic tool (300). The size of the donut-shaped light spot (S1, S2) varies depending on the distance between the endoscopic tool (i.e., the tip of the endoscopic tool) and the surface of the tissue inside the human body. The setting unit (555) pre-sets the standard distance between the endoscopic tool and the surface of the tissue inside the human body according to the size of the donut-shaped single light spot, and this standard distance may be stored in the memory unit (552) as a look-up table. The calculation unit (555) can accurately calculate the distance between the endoscopic tool and the tissue surface by comparing the value measured by the size measurement unit (551a) for the size of the donut-shaped light spot formed on the tissue surface.

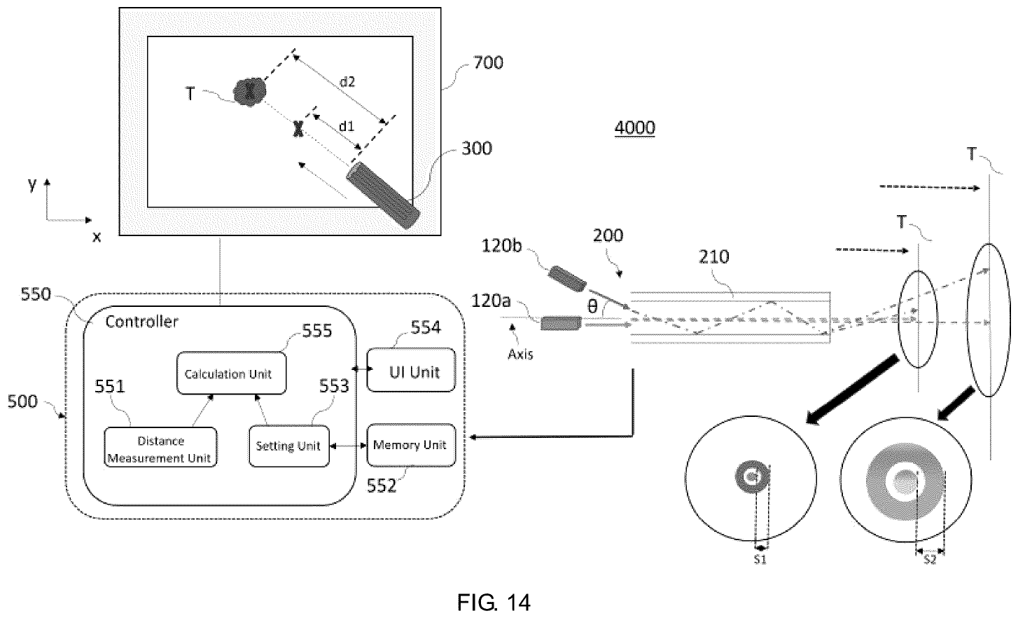

FIG. 14 shows a schematic diagram of an endoscope system according to a fourth embodiment of the present disclosure.

As shown, the endoscope system (4000) according to the fourth embodiment includes the same or similar components and effects as the endoscope system (1000) according to the first embodiment.

However, in the embodiment, the light guide unit (200) may include two single light sources (120a, 120b) for irradiating single light into the interior of the optical fiber (210), and the optical fiber (210) may be composed of multiple fibers in a bundle form. In this case, the first light source (120a) may be arranged parallel to the axial axis (Axis) of the optical fiber (210), and the second light source (120b) may be arranged at a predetermined angle (0) with respect to the axial axis (Axis) of the optical fiber (210). The first and second light sources may be composed of different colors. The light guide unit (200) having such a structure forms a light spot on the surface of the tissue inside the human body as it is operated by the operator, and forms a donut-shaped light spot on the surface of the tissue inside the human body from the second light source (120b). The distance (S1, S2) between the light spot and the donut-shaped light spot formed on the tissue surface is larger as the distance between the optical fiber and the tissue surface increases.

The control unit (550) may include a distance measurement unit (551), a setting unit (553), and a calculation unit (555). The distance measurement unit (551) calculates the distance between the light spot and the donut-shaped light spot formed on the tissue surface by the light irradiated from the light guide unit (200) included in the endoscopic tool (300). The distance between the light spot and the donut-shaped light spot (S1, S2) varies depending on the distance between the endoscopic tool (i.e., the tip of the endoscopic tool) and the surface of the tissue inside the human body. The setting unit (555) pre-sets the standard distance between the endoscopic tool and the surface of the tissue inside the human body according to the distance between the light spot and the donut-shaped light spot, and this standard distance may be stored in the memory unit (552) as a look-up table. The calculation unit (555) can accurately calculate the distance between the endoscopic tool and the tissue surface by comparing the value measured by the distance measurement unit (551) for the distance between the light spot and the donut-shaped light spot formed on the tissue surface according to the movement of the endoscopic tool with the pre-set standard distance value from the setting unit (555).

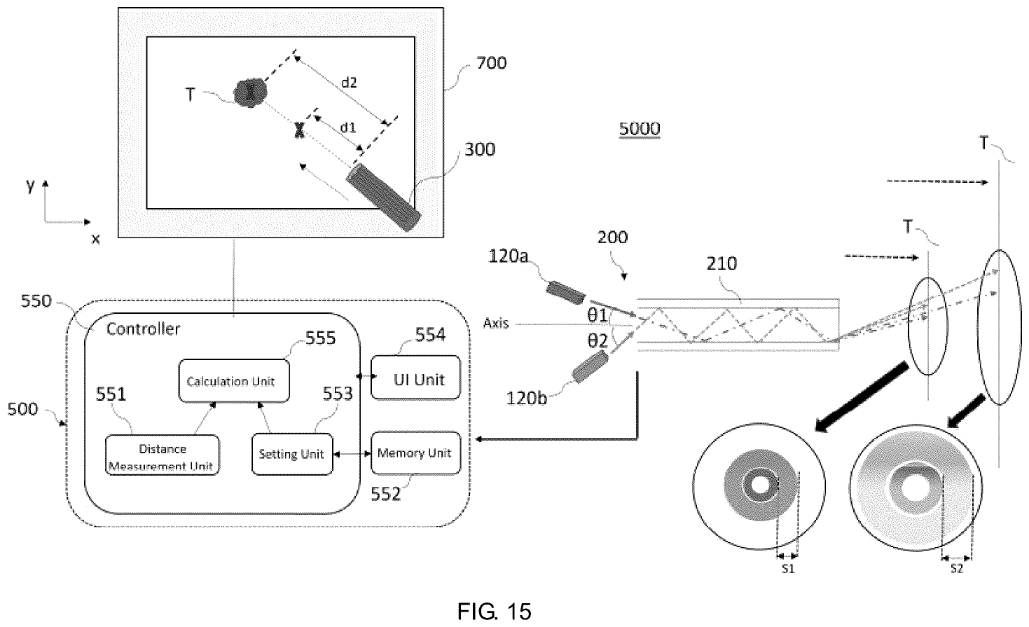

FIG. 15 shows a schematic diagram of an endoscope system according to a fifth embodiment of the present disclosure.

As shown, the endoscope system (5000) according to the fifth embodiment includes the same or similar components and effects as the endoscope system (1000) according to the first embodiment.

However, in the embodiment, the light guide unit (200) may include two single light sources (120a, 120b) for irradiating single light into the interior of the optical fiber (210), and the optical fiber (210) may be composed of multiple fibers in a bundle form. In this case, the first light source (120a) may be arranged at a predetermined angle (01) with respect to the axial axis (Axis) of the optical fiber (210), and the second light source (120b) may be arranged at a predetermined angle (02) greater than the first angle with respect to the axial axis (Axis) of the optical fiber (210). The first and second light sources may be composed of different colors. The light guide unit (200) having such a structure forms a first donut-shaped light spot on the surface of the tissue inside the human body as it is operated by the operator, and forms a second donut-shaped light spot on the surface of the tissue inside the human body from the second light source (120b). The distance (S1, S2) between the first donut-shaped light spot and the second donut-shaped light spot formed on the tissue surface is larger as the distance between the optical fiber and the tissue surface increases.

The control unit (550) may include a distance measurement unit (551), a setting unit (553), and a calculation unit (555). The distance measurement unit (551) calculates the distance between the first donut-shaped light spot and the second donut-shaped light spot formed on the tissue surface by the light irradiated from the light guide unit (200) included in the endoscopic tool (300). The distance between the first donut-shaped light spot and the second donut-shaped light spot (S1, S2) varies depending on the distance between the endoscope instrument (the tip of the endoscope instrument) and the surface of the tissue inside the human body. The setting unit (555) pre-sets the standard distance between the endoscopic tool and the surface of the tissue inside the human body according to the distance between the first donut-shaped light spot and the second donut-shaped light spot, and this standard distance may be stored in the memory unit (552) as a look-up table. The calculation unit (555) can accurately calculate the distance between the endoscopic tool and the tissue surface by comparing the value measured by the distance measurement unit (551) for the distance between the first donut-shaped light spot and the second donut-shaped light spot formed on the tissue surface according to the movement of the endoscopic tool with the pre-set standard distance value from the setting unit (555).

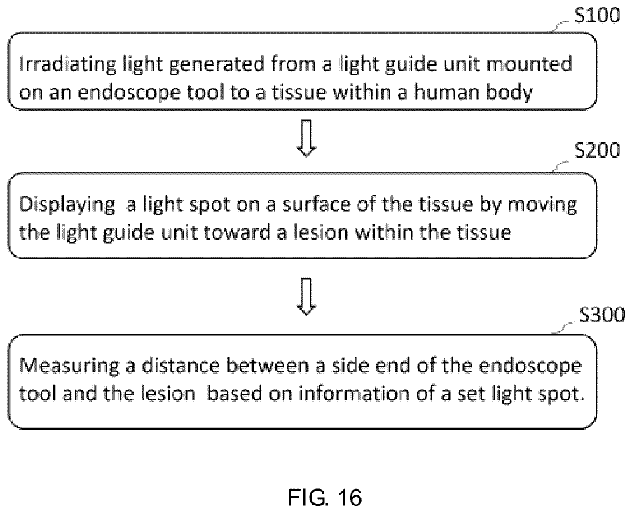

FIG. 16 is a flowchart to explain a method for determining a location of a lesion using an endoscope system according to embodiments of the present disclosure.

As shown in FIG. 16, in step S100, light emitted from the light guide unit mounted on the endoscope tool is irradiated to the tissue inside the human body. In this case, the light irradiated to the tissue may be a single or multiple light. Subsequently, in step S200, the light guide unit is moved in the direction of the lesion in the tissue so that the focus of the irradiated light is targeted to the lesion in the tissue. As a result, the light targeted to the lesion in the tissue forms a light spot on the surface of the tissue. At this time, the light spot may be multiple or single depending on the number of lights. In step S300, the distance between the tip of the endoscope tool and the lesion in the tissue is measured based on the information of the set light spot. In this case, the information of the light spot is the pre-set distance from the endoscope tool to the lesion in the tissue according to the distance between the light spots when the light spot is multiple, and the pre-set distance from the endoscope tool to the lesion in the tissue according to the size of the light spot when the light spot is single.

The shape of the light spot may be a circular or donut-shaped circle, or it may have a variety of other pattern shapes.

By measuring the distance between the endoscope tool and the lesion in the tissue in this way, the accurate location of the lesion inside the tissue can be determined. As a result, it becomes possible to accurately identify the location of the lesion when using an endoscope device for tissue examination, and to remove or resect the lesion tissue.

As described above, it has been reviewed a desirable embodiment according to the present disclosure. It is self-evident to those with ordinary knowledge of the relevant technology that the present invention can be concretized in other specific forms without deviating from its purpose or category, in addition to the embodiments described above. Therefore, the aforementioned embodiments should be considered to be illustrative rather than restrictive, and accordingly, the present invention may be modified within the scope of the attached claim and its equivalent range without being limited to the explanation mentioned above.

1. An endoscopic tool provided at the distal end of an endoscope device, comprising:
a main body capable of operating by an operator; and
a light guide unit provided at the main body to emit light in a direction in which the main body moves.

2. The endoscopic tool of claim 1,
wherein the light guide unit includes a light source generating light, a housing fixed to a preset position on the main body, and a cable that has one side connected to the light source and the other side connected to an external power source to supply power to the light source.

3. The endoscopic tool of claim 1,
wherein the light guide unit includes a light source generating light, a housing fixed to a preset position on the main body, and a power supply placed within the housing and connected electrically to the light source to supply power to the light source.

4. An endoscope system comprising:
an endoscope device having a tube in which a camera is placed in one of a plurality of channels formed inside the endoscope device;
an endoscopic tool having a light guide unit that is inserted into another channel among the plurality of channels and irradiates a plurality of lights so that at least two light spots are focused on a surface of a tissue inside a human body; and
a controller measuring a distance from a tip of the endoscopic tool to the surface of the tissue according to a distance between light spots formed on the surface of the tissue based on a preset distance from the tip of the endoscopic tool to the surface of the tissue according to the distance between light spots.

5. The endoscope system of claim 4,
wherein the light guide unit includes a plurality of light sources that generate a plurality of lights, and
an optical fiber that is optically connected to the plurality of light sources and guides the plurality of lights to be totally reflected inside the optical fiber and to be irradiated to the surface of the tissue.

6. The endoscope system of claim 5,
wherein the plurality of light sources is arranged at different angles with respect to an input end of the optical fiber to irradiate the light into the optical fiber.

7. The endoscope system of claim 5,
wherein the optical fiber is formed by a single piece.

8. The endoscope system of claim 4,
wherein the plurality of lights is emitted from the light guide unit at a predetermined angle that is not parallel to each other.

9. The endoscope system of claim 4, further comprising a display unit that displays the surface of the tissue through the camera;
wherein a trace mark according to a movement of the endoscopic tool is displayed on a effective screen of the display unit based on the measured distance.

10. An endoscope system comprising:
an endoscope device having a tube in which a camera is placed in one of a plurality of channels formed inside the endoscope device;
an endoscopic tool having a light guide unit that is inserted into another channel among the plurality of channels and irradiates light so that a light spot is focused on a surface of a tissue inside a human body; and
a controller measuring a distance from a tip of the endoscopic tool to the surface of the tissue according to a size of the light spot formed on the surface of the tissue based on a preset distance from the tip of the endoscopic tool to the surface of the tissue according to the size of the light spot.

11. The endoscope system of claim 10,
wherein the light guide unit includes a light source that generates light, and an optical fiber that is optically connected to the light source and guides the light to be totally reflected inside the optical fiber and to be irradiated to the surface of the tissue.

12. The endoscope system of claim 11,
wherein the light source irradiates the light to an input end of the optical fiber in parallel with a longitudinal axis of the optical fiber or irradiates the light to the input end of the optical fiber at a predetermined angle with respect to the longitudinal axis of the optical fiber.

13. A method for determining a location of a lesion, comprising;
forming a light spot on a surface of a tissue by irradiating light to the tissue inside a human body using a light guide unit mounted on an endoscopic tool; and
measuring a distance between a tip of the endoscopic tool and the lesion of the tissue based on information of a preset light spot.

14. The method of claim 13,
wherein if the light spot is plural, the information of the preset light spot is a preset distance from the tip of the endoscopic tool to the lesion of the tissue according to a distance between the light spots.

15. The method of claim 13,
wherein if the light spot is a single, the information of the preset light spot is a preset distance from the tip of the endoscopic tool to the lesion of the tissue according to a size of the light spot.
